(19) United States
(12) Patent Application Publication
(10) Pub. No.: US 2005/0160799 A1
(43) Pub. Date: Jul. 28, 2005

(54) Analytical method and apparatus for analysis for surfactants in hydrocarbon distillate fuels

(76) Inventor: Spencer Edwin Taylor, Camberley (GB)

Correspondence Address: CAROL WILSON;BP AMERICA INC., MAIL CODE 5 EAST, 4101 WINFIELD ROAD, WARRENVILLE, IL, 60555, US

(21) Appl. No.: 10/499,086

(22) PCT Filed: Nov. 28, 2002
(86) PCT No.: PCT/GB02/05365

(30) Foreign Application Priority Data
Dec. 19, 2001 (GB) 0130287.6

(51) Int. Cl. G01N 33/22; G01N 1/10

(52) U.S. Cl. 73/61.62; 73/61.41; 73/61.43; 73/64.56

(57) ABSTRACT

A method and apparatus for determining the content of the surfactant in a hydrocarbon distillate fuel wherein: (a) a wettable surface is contacted with a fuel comprising at least one surfactant, (b) the fuel and non adsorbed surfactant is separated from the wettable surface to leave a deposit of the surfactant upon the wettable surface, (c) the wettable surface comprising the surfactant deposit is then exposed to a wetting liquid, (d) the extent or rate of displacement of the wetting liquid over the wettable surface comprising the surfactant deposit is measured, and (e) said measurement is converted to provide a value for the content of surfactant in the hydrocarbon distillate fuel.

The present invention relates to methods of analysis for surfactants in hydrocarbon distillate fuels, especially jet fuels.

In high speed aircraft, both civilian and military, the liquid fuel is combusted to produce power, but also is circulated in the aircraft as a heat exchange fluid to remove the excess heat generated at such speeds e.g. in lubricating oils. The fuel is thus maintained for long periods at high temperatures, which results in discoloration and decomposition to produce soluble and insoluble products such as gums, sediments and granular material. The insoluble products can form deposits that reduce the heat exchange capacity and can block filters, potentially causing loss of power. Consequently jet fuels usually contain conventional additives such as antioxidants, corrosion inhibitors, dispersants and detergents to reduce the extent of decomposition and suspend decomposition products in the body of the fuel therefore preventing blockages by preventing deposition.

However some of the dispersant and detergent additives are surface-active materials and have been implicated for many years in problems when water is present in the fuels. In addition to the presence of conventional additives, further surface-active materials may also be introduced into the jet fuel in the form of contaminants. If the fuel has an undesirable content of surface-active materials, this can lead to the formation of stable finely-dispersed water-in-oil emulsions, on the one hand, whilst modifying the surfaces of filter-coalescers designed to remove suspended water and solids, on the other. Either way, water retention by the fuel is increased with undesirable consequences.

The levels of surfactants within the fuel need to be determined to enable the identification of any undesirably high levels of surfactant content which would consequently cause adverse effects during water separation. Examples of such surfactants include antistatic additives, DiEGME (diethylene glycol monomethyl ether) as an icing inhibitor, salicylidene derivatives as a metal deactivator, and phenols, indoles and sulphonates as contaminants.

The existing method of determining the levels of surfactants in fuels uses a so-called microseparometer that uses expensive, single-analysis disposable coalescence cells. The method involves the mixing an amount of fuel with water to form a dispersion under standard conditions, which is subsequently passed through a coalescer to remove large droplets. The turbidity of the dispersion remaining is then measured and this provides a measure of the surfactant content of the fuel. This method is not only expensive and time consuming but must be carried out by a person skilled in the art under laboratory conditions.

There has now been discovered a method of determining the surfactant composition of hydrocarbon distillate fuels which comprises the measurement of the extent or rate of displacement of the solvent front of a wetting liquid over a wettable surface that has been exposed to the surfactant-containing fuel. The extent or rate of displacement of the solvent front is not limited to a displacement in any one direction, so it can be linear or radial displacement, in a direction which may be horizontal or vertical. The wettability of a surface can be determined by monitoring the time it takes for the solvent front of a wetting liquid to travel a predetermined distance over a wettable surface. The wettability of the surface with the specific fuel may then be compared to wettabilities from standard fluids containing surfactant.

Accordingly, the present invention provides a method of determining the content of surfactant in a hydrocarbon distillate fuel wherein:
a) a wettable surface is contacted with a fuel comprising at least one surfactant resulting in the uptake of surfactant onto said surface,
b) the fuel and any non uptaken surfactant are separated from the wettable surface to leave a deposit of the uptaken surfactant upon the wettable surface,
c) the wettable surface comprising the surfactant deposit is then exposed to a wetting liquid,
d) the extent or rate of displacement of the wetting liquid over the wettable surface comprising the surfactant deposit is measured, and
e) said measurement is converted to provide a value for the content of surfactant in a hydrocarbon distillate fuel.

The present invention also provides a substrate that comprises a wettable surface. The surface of the substrate may possess sufficient wettable character for use in the method of the invention, or alternatively the substrate surface can be modified with an applied wettable coating. The substrate and the wettable coating are inert and stable when exposed to the hydrocarbon distillate fuel. The wettable surface is preferably water wettable and preferably has a wettability limit defined in terms of a respective lower and upper contact angle limit of 20-80 e.g. 30-60 degrees.

The wettable surface has a shape sufficient to allow the measurement of the displacement of the solvent front of the wetting liquid over it. The wettable surface may be flat i.e. linear-in 2 directions-at-right angles-or-curved but linear in 1 direction. The wettable surface is usually the internal surface area of a substrate tube wherein the substrate tube and any applied wettable coating are at least in part transparent to enable the monitoring of the displacement of the solvent front of the wetting liquid over the wettable surface.

The wettable surface may also be provided by the external surface area of a substrate rod, plate or a disc. One or preferably both of the substrate and any applied wettable coating are preferably at least in part transparent; however both the substrate and the wettable coating can be opaque, especially when used in conjunction with a wetting liquid which contains a small amount of dye. The dye then allows the displacement of the solvent front of the wetting liquid over the wettable surface to be monitored.

The tubes and rods usually have radius of 0.1-10 mm, in particular 0.5-5 mm e.g. 1 mm, and the tubes, rods, and plates usually have a length sufficient to accommodate a movement of wetting liquid of between 1-100 mm, in particular 5-50 mm, and especially 10-20 mm. The discs usually have a radius sufficient to accommodate a movement of wetting liquid of between l-100 mm, in particular 5-50 mm, and especially 10-20 mm. The rod or tube can be arranged in a flat coil providing a practical, more convenient piece of apparatus capable of measuring large displacements.

Typical compounds suitable for use as wettable surfaces are alumina, silica, hydrophobised silica, glass/polymer fibres and “Perspex” polymer and as such these compounds can be either used as the substrate or alternatively applied as a wettable coating to a substrate in which case alkoxy silanes with polar side chains, such as Me₂N propyl Si(OMe)₃ may also be used. The substrates are typically glass, quartz or “Perspex” polymer. The wettable surfaces exhibit at least one and especially two marks wherein the rate of displacement of the solvent front of the wetting liquid between said marks could be measured. Preferably the wettable surface is graduated.

However a further embodiment of the invention provides an electrically conducting tubular substrate e.g. graphite but preferably metal wherein at least part of the internal surface area of which comprises a wettable coating. The progress of the wetting liquid over the wettable coating may be monitored with the aid of an electrical circuit such that as the wetting liquid reaches a pre-designated displacement the circuit is completed and some method of indication-of this can be provided e.g. alight or a bell, and the time taken to attain the displacement is noted either manually or electronically. The rate of displacement of the wetting liquid can then be calculated taking into account the time taken to attain such a displacement. Consequently this can then be related to the surfactant composition of the fuel.

A thin layer e.g. 1-10 microns thick of a wettable coating can be applied onto the substrates to provide the wettable surfaces using dip coating for rods, tubes, discs, and plates or spin coating for plates and discs; for dip or spin coating the substrate is usually treated with the coating agent in the form of a sol-gel or a suspension. Chemical vapour deposition, wherein the wettable coating or precursor thereof, is carried out in vapour form via an inert gas to the substrate, or alternatively treating the substrate with vapour under vacuum is particularly useful in coating the internal surface area of tubular substrates.

Different wettable surfaces possess different affinities for the different types of surfactant, and therefore acidic, basic and non-polar surfaces can be used so as to differentiate the attraction towards anionic, cationic, non-ionic, and ampholytic surfactants. Preferably the fuel is analysed using the three different types of wettable surfaces i.e. acidic (e.g. alumina), basic (e.g. Me₂N propyl Si(OMe)₃) or neutral (e.g. hydrophobicised silica). Employing the three surfaces for analysis of the fuel provides a fingerprint of the concentration of each type of surfactant within that fuel.

Accordingly, a preferred embodiment of the invention provides an analytical test unit which comprises at least one substrate and at least two wettable surfaces, preferably three wettable surfaces, each wettable surface on a substrate wherein the wettable surfaces possess different degrees of acid, neutral and/or base character. Preferably, the three wettable surfaces are provided by internal surface areas of three tubular substrates wherein one substrate possesses an acidic wettable surface, one substrate possesses a basic wettable surface and one substrate possesses a neutral wettable surface. Preferably, the analytical test unit comprises a support means to which the substrates are attached in substantially the same orientation, e.g. substantially normal to an elongate support.

Alternatively, the unit may comprise an elongated substrate having at least two, preferably at least three, elongate wettable surfaces of different character e.g. a bar having 2-4 sides e.g. 3 wherein one side possesses an acidic wettable surface, one side possesses a basic wettable surface and one side possesses a neutral wettable surface.

The preferred embodiment of the invention provides a field analysis kit which comprises:
a) an analysis fuel container with a volume of typically 10-1000 ml in particular 250-750 ml e.g. 500 ml for containing an aliquot of analysis fuel,
b) a wetting liquid container with a volume of typically 10-1000 ml in particular 250-750 ml e.g. 500 ml for containing a reservoir of wetting liquid,
c) a lid capable of acting as a closure means for one or both of said containers wherein an analytical test unit as described herein above is supported by said lid such that it hangs substantially vertically downwards,
d) and optionally a source of a gas stream e.g. a canister of compressed gas or air.

The analysis fuel and the wetting liquid containers may be provided by 2 separate containers that are preferably releaseably connected. In this case there may be two lids each capable of acting as a closure means for the two containers individually or alternatively, particularly when the containers are connected, one lid may be present capable of acting as a closure means for both containers simultaneously.

In another preferred embodiment the analysis fuel and the wetting liquid containers are provided by one container that is subdivided into two chambers by a partition and in this case the lid is preferably capable of acting as a closure means for both chambers simultaneously.

Mountings on the inside of the lid may be provided so that the lid is capable of removably receiving the substrates of the analytical test unit. Preferably one or more orifices e.g. slots may be present in the lid through which the substrate(s) can extend 5 enabling the required contact of the substrates with analysis fuel and then the wetting liquid.

In the method of the present invention, the wettable surface(s) is/are contacted by the analysis fuel comprising at least one surfactant preferably with the contact time sufficient to allow the uptake of the surfactant onto the wettable surface to attain equilibrium. Standard conditions are employed in order to achieve this and to attain maximum reproducibility. Typically, a known aliquot of fuel of 10-1000 ml in particular 250-750 ml e.g. 500 ml is contacted with the wettable surface. Typically, the contacting of the fuel with the wettable surface is performed at room temperature. Typically, the fuel is contacted with the wettable surface(s) for 1-30 min, e.g. 2-10 min e.g. 5 min. Preferably, a known aliquot of fuel of 10-1000 ml in particular 250-750 ml e.g. 500 ml is contacted at room temperature with the wettable surface(s) for 1-30 min, e.g. 2-10 min e.g. 5 min. Preferably, exposure of the wettable surface to the surfactant is achieved by passage of or preferably by continuous recirculation of the given aliquot of analysis fuel over the wettable surface e.g. passing it through a tube having the surface on the inside. This can be achieved with the aid of a pump driven by an electric motor. Alternatively, the wettable surface can be submersed in the given aliquot of analysis fuel and preferably the fuel is agitated by employing an ultrasonic or a stirred tank.

Advantageously, the wettable surface and the aliquot of analysis fuel are contacted in a substantially closed container to avoid evaporation of the fuel. This may be achieved when using the field analysis kit of the invention by placing the lid comprising the analytical test unit on having the test unit extending therethrough on top of the analysis fuel container/chamber such that analytical test unit is submersed in the aliquot of analysis fuel.

The fuel is then separated from the wettable surface so that a uniform deposition of surfactant upon the wettable surface is achieved. Preferably this is done by draining the majority of the fuel from the substrate and evaporating the remaining fuel from the wettable surface for example by placing the substrate carrying the wettable surface in a rotary evaporator at room temperature under vacuum for typically 10-120 mins e.g. 60 mins or most preferably in a stream of gas e.g. air, which can be provided by the canister of compressed gas e.g. air when using the field analysis kit of the invention.

The wettable surface comprising the surfactant is then exposed to the wetting liquid. Where the substrate is a tube, plate or rod this can be achieved by positioning the substrate substantially upright and placing the bottom of the substrate in a reservoir containing the wetting liquid. Advantageously the substrate and reservoir are in a substantially closed container to avoid evaporation of the wetting liquid. This is achieved when using the field analysis kit by placing the lid comprising the analytical test unit on having the test unit extending therethrough on top of the wetting liquid container/chamber such that the bottom of the analytical test unit is contacted with the reservoir of wetting liquid.

Preferably, the wettable surface is water wettable and the wetting liquid is water though other examples of wetting liquids are ethylene glycol, ethanol and methanol.

Where the substrate is a disc, the disc can be positioned substantially upright whilst the wetting liquid is supplied via a pipe connected to a reservoir to the centre of the disc. The disc can be optionally rotated about its vertical axis.

The extent or rate of displacement of the wetting liquid over the wettable surface comprising the surfactant deposit may be measured by measuring the rate or extent of the vertical displacement (in the case of tubes, rods and plates) or radial displacement (in the case of discs) of the solvent front of the wetting liquid.

Preferably, the rate of displacement is calculated by measuring the time taken for the solvent front of the wetting liquid to pass between two standard points on the wettable surface.

The rate of displacement is then directly or indirectly converted to give a value for the content of a type of surfactant present in the fuel. The rate of displacement can be converted into a contact angle (taking into account the surface tension and viscosity of the wetting liquid) and applying the Washburn equation which relates the height (h) risen by a solvent front to the time taken (t), such that
h²=rγt cos θ/2η
where γ is the surface tension of the wetting liquid, θ is the contact angle, η the viscosity of the wetting liquid, and r is a constant for a given wettable surface.

Determination of constant r for the wettable surfaces can be carried out by use of a standard (hydrocarbon) liquid (or liquids) for which θ is zero and γ and η are known. θ can then be obtained from the respective parameter set for water.

The contact angle or the extent or rate of displacement can then be used as a characteristic measurement of the levels of the different types of surfactant in the fuel by comparison with calibration standards.

Surfactants with an acidic character will be primarily deposited upon a basic wettable surface, a wettable surface with acidic character will primarily attract surfactants with basic characteristics, whilst a neutral wettable surface will adsorb most surfactants. The deposition of the surfactants will affect the wettability of the surfaces and consequently the rate of displacement. The adsorption of the surfactants may increase or decrease the wettability of the surface depending on the nature of the interaction and surfactants present.

Calibration relationships can be produced by plotting either the rate or extent of displacement or contact angle (calculated from the Washburn equation) with a particular wettable surface under standard conditions, the surface having been exposed to distillate fuel containing varying known concentrations of surfactant against the concentration of surfactant in the fuel. The varying known concentrations of surfactant in the fuel are primarily determined by conventional methods e.g. chromatographic analysis (a microseparator). Thus calibration relationships can be produced for the following systems: acidic surfactants—basic wettable surfaces, basic surfactants—acidic wettable surfaces, and neutral surfactants—neutral wettable surfaces.

Therefore, using the three types of wettable surfaces in combination provides a simple field analysis kit with the means of characterising the type and concentration of each type of surfactant material contained in the analysis fuel. The analysis kit provides a cheap and robust method that is easy to operate and produces results that are easily interpreted in the field.

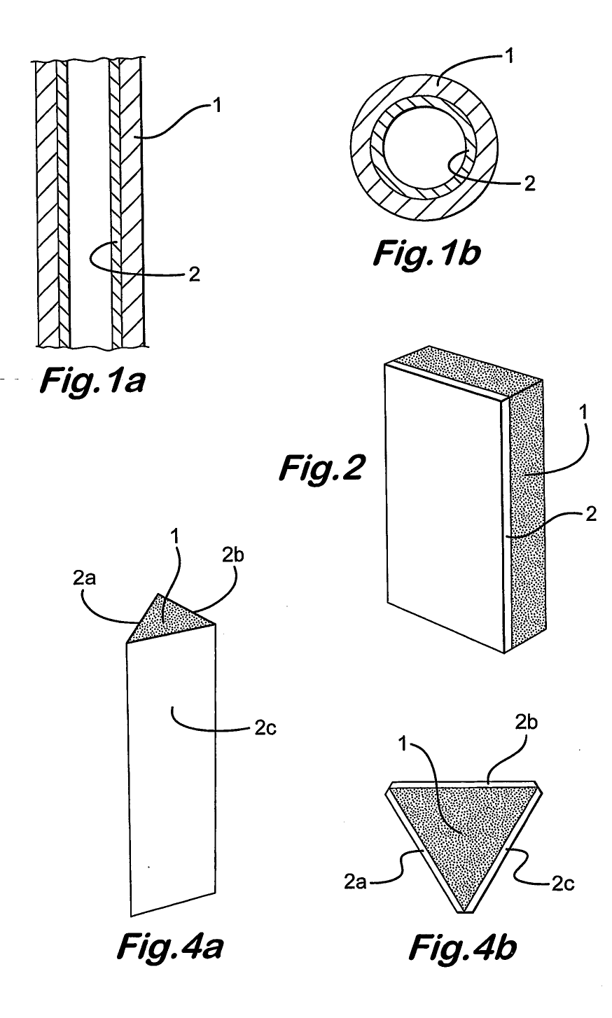

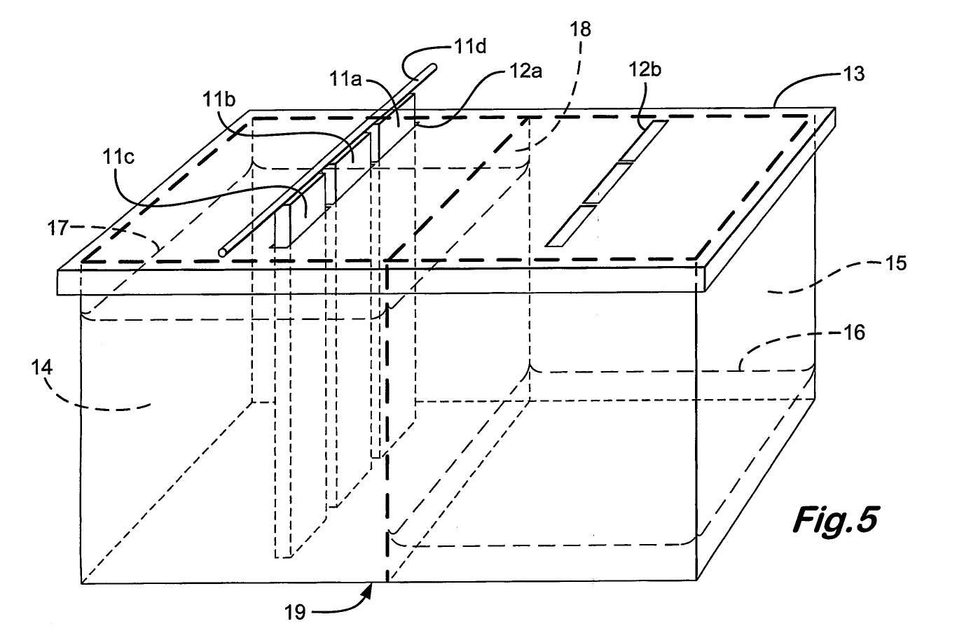

The invention will now be described with reference to the accompanying drawings and is illustrated in the following examples in which FIGS. 1 to 4 represent perspective, and cross section views of substrates according to the present invention and FIG. 5 represents a field test kit according to the present invention.

FIG. 1a represents a longitudinal cross section of a tube comprising a substrate (1) having an internal wettable surface (2). FIG. 1b is a radial cross section of 1a.

FIG. 2 represents a view of a plate comprising a substrate (1) having an external wettable surface (2).

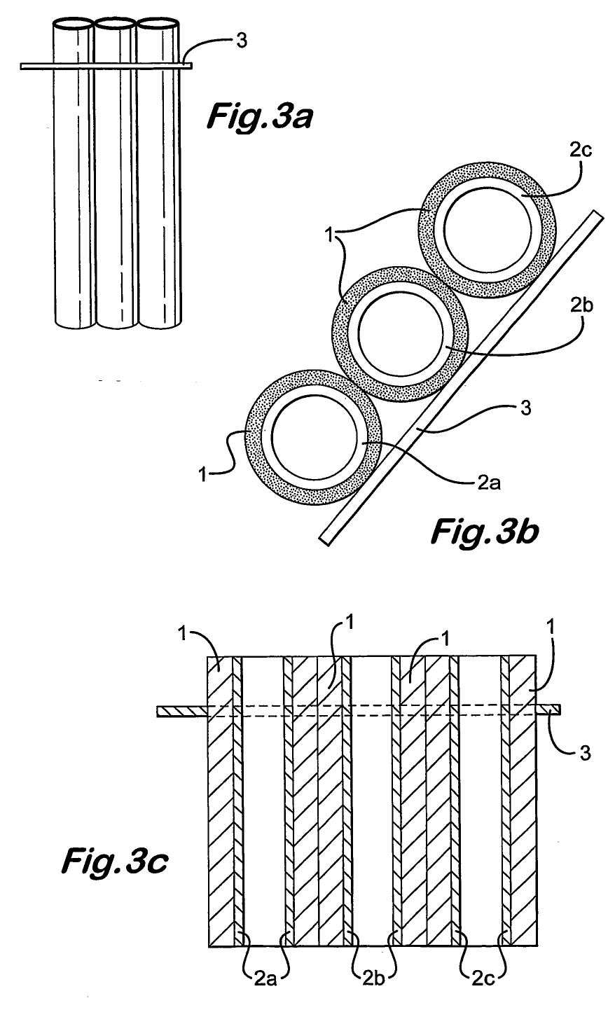

FIG. 3a shows a representation of an analytical test unit comprising three tubes as shown in FIGS. 1a and 1b and a support means (3) e.g. in the form of a transverse plate integral with or adhered to said substrate tubes or plates. FIG. 3b is a plan view of the analytical test unit with three tubes of substrate (1), the tubes carrying respectively (2a) an acidic wettable surface, (2b) a basic wettable surface and (2c) a neutral wettable surface. FIG. 3c represents a longitudinal cross section of the analytical test unit.

FIG. 4a shows a representation of a second analytical test unit comprising a triangular rod of substrate (1) having respectively on its three longitudinal sides an acidic wettable surface (2a), a basic wettable surface (2b) and a neutral wettable surface (2c). FIG. 4b is a plan view of the analytical test unit.

The substrates illustrated in FIGS. 1 to 4 may be used in the method of the present invention method to determine the content of surfactant in a hydrocarbon distillate fuel wherein:
a) a wettable surface is contacted with a fuel comprising at least one surfactant resulting in the uptake of surfactant onto said surface,
b) the fuel and non adsorbed surfactant is separated from the wettable surface to leave a deposit of the surfactant upon the wettable surface,
c) the wettable surface comprising the surfactant deposit is then exposed to a wetting liquid,
d) the extent or rate of displacement of the wetting liquid over the wettable surface comprising the surfactant deposit is measured,
e) said measurement is converted to provide a value for the content of surfactant in the hydrocarbon distillate fuel.

FIG. 5 shows a representation of a field analysis kit which comprises an analytical test unit constructed from three substrate plates (11a), (11b) and (11c) such as those illustrated in FIG. 2 and a support means (11d), an analysis fuel container and a wetting liquid container provided by one container (19) which is subdivided into two chambers (14) and (15) by a partition (18) and a lid (13) capable of acting as a closure means for both chambers simultaneously wherein slots (12a) through which the substrates can extend enabling contact with the analysis fuel (17) and slots (12b) through which the substrates can extend enabling the contact with the wetting liquid (16) are provided.

The field test kit may also be used in the method according to the present invention.

1. A method of determining the content of surfactant in a hydrocarbon distillate fuel wherein:
a) a wettable surface is contacted with a fuel comprising at least one surfactant resulting in the uptake of surfactant onto said surface,
b) the fuel and non adsorbed surfactant is separated from the wettable surface to leave a deposit of the surfactant upon the wettable surface,
c) the wettable surface comprising the surfactant deposit is then exposed to a wetting liquid,
d) the extent or rate of displacement of the wetting liquid over the wettable surface comprising the surfactant deposit is measured,
e) said measurement is converted to provide a value for the content of surfactant in the hydrocarbon distillate fuel.

2. A method as claimed in claim 1 in which a known aliquot of fuel of 10-1000 ml in particular 250-750 ml e.g. 500 ml is contacted with the wettable surface at room temperature for 1-30 min, e.g. 2-10 min e.g. 5 min.

3. A method as claimed in claim 1 in which the fuel is passed over or continuously recirculated over the wettable surface.

4. A method as claimed in claim 1 in which the wettable surface is submersed in an aliquot of the fuel and preferably the fuel is agitated by employing an ultrasonic or a stirred tank.

5. A method as claimed in claim 1 in which the wettable surface is contacted with an aliquot of fuel in a substantially closed container.

6. A method as claimed in claim 1 in which the fuel is separated from the wettable surface by draining the majority of the fuel from the substrate and evaporating the remaining fuel from the wettable surface.

7. A method as claimed in claim 1 in which the wettable surface comprising the surfactant is exposed to the wetting liquid by positioning the substrate substantially upright and placing the bottom of the substrate in a reservoir containing the wetting liquid.

8. A method as claimed in claim 1 in which the wettable surface is water wettable and the wetting liquid is water.

9. A method as claimed in claim 1 in which the rate of displacement of the wetting liquid over the wettable surface comprising the surfactant is calculated by measuring the time taken for the solvent front of the wetting liquid to pass between two standard points on the wettable surface.

10. A method as claimed in claim 1 in which the wettable surface is selected from a group consisting of acidic, basic and neutral surfaces.

11. A method as claimed in claim 1 in which the surfactant content in the fuel is determined using at least three different wettable surfaces.

12. A method as claimed in claim 11 in which the three different wettable surface comprise a basic wettable surface, an acidic wettable surface and a neutral wettable surface.

13. A substrate for use in a method as claimed claim 1, which substrate is a tube having an inert wettable surface which is at least part of the internal surface of said tube.

14. A substrate according to claim 13 in which the tube and any applied wettable coating is transparent at least in part.

15. A substrate for use in a method as claimed in claim 1 in which the substrate is selected from the group consisting of a rod, plate and a disc and the wettable surface is the external surface area of the substrate.

16. A substrate as claimed in claim 15 in which the wettable surface is opaque.

17. A substrate as claimed in claim 13 in which said surface is a wettable coating on said substrate.

18. A substrate as claimed in claim 13 in which the wettable surface is water wettable.

19. A substrate as claimed in claim 13 in which the wettable surface is selected from a group consisting of acidic, basic and neutral surfaces.

20. A substrate as claimed in claim 19 in which the acidic wettable surface is alumina, the basic wettable surface comprises Me₂N propyl Si(OMe)₃ and the neutral wettable surface is hydrophobicised silica.

21. An analytical test unit that comprises at least one substrate as claimed in claim 13 and at least two wettable surfaces which possess different acid-base character.

22. A unit as claimed in claim 21 in which said unit comprises an acidic, basic and neutral wettable surface.

23. A unit as claimed in claim 22 which comprises at least three tubular substrates wherein one substrate possesses an acidic wettable surface, one substrate possesses a basic wettable surface and one substrate possesses a neutral wettable surface.

24. A unit as claimed in claim 23 in which the wettable surfaces are provided by the internal surface areas of the tubular substrates.

25. A unit as claimed in claim 22 which comprises a triangular rod substrate wherein one side possesses an acidic wettable surface, one side possesses a basic wettable surface and one side possesses a neutral wettable surface.

26. A field test unit which comprises:
a) an analysis fuel container with a volume of typically 10-1000 ml in particular 250-750 ml e.g. 500 ml for containing an aliquot of analysis fuel,
b) a wetting liquid container with a volume of typically 10-1000 ml in particular 250-750 ml e.g. 500 ml for containing a reservoir of wetting liquid,
c) a lid capable of acting as a closure means for one or both of said containers wherein an analytical test unit as claimed in claim 21 is supported by said lid such that it hangs substantially vertically downwards, and optionally a source of a gas stream e.g. a canister of compressed gas or air.

27. A method as claimed in claim 1 in which the wettable surface is contacted with an aliquot of fuel in a substantially closed container; the fuel is separated from the wettable surface by draining the majority of the fuel from the substrate and evaporating the remaining fuel from the wettable surface; the wettable surface comprising the surfactant is exposed to the wetting liquid by positioning the substrate substantially upright and placing the bottom of the substrate in a reservoir containing the wetting liquid; and the rate of displacement of the wetting liquid over the wettable surface comprising the surfactant is calculated by measuring the time taken for the solvent front of the wetting liquid to pass between two standard points on the wettable surface.

28. A method as claimed in claim 1 in which the wettable surface is water wettable, the wetting liquid is water and the surfactant content in the fuel is determined using at least three different wettable surfaces.
